(19) United States
(12) Patent Application Publication
(10) Pub. No.: US 2012/0285459 A1
(43) Pub. Date: Nov. 15, 2012

(54) AIR DISINFECTION AND CLEANING DEVICE, AND EXHALED GAS DISINFECTION AND CLEANING DEVICE, INTERIOR AIR DISINFECTION AND CLEANING DEVICE, AND SIMPLIFIED ISOLATION DEVICE USING THE SAME

(76) Inventors: Yasuhiko Sata, Tokyo (JP); Norio Hachisu, Tokyo (JP)

(21) Appl. No.: 13/521,570

(22) PCT Filed: Jan. 14, 2011
(86) PCT No.: PCT/JP2011/050575

§ 371 Date: Jul. 11, 2012

(30) Foreign Application Priority Data
Jan. 15, 2010 (JP) 2010-007450

(51) Int. Cl. A61L 2/10; A62B 23/02; A61M 16/00

(52) U.S. Cl. 128/205.12; 250/436; 128/205.27

(57) ABSTRACT

An air disinfection and cleaning device is provided that includes: a cylindrical reflector having a cylindrical inner surface in which an air flows from an upper end to a lower end and the cylindrical inner surface is subjected to mirror finishing; a rod shaped ultraviolet lamp disposed at the center within the cylindrical reflector parallel to the long side; photocatalytic sheet filters being air permeable photocatalytic sheet filters having a diameter nearly equal to an inner diameter of the cylindrical reflector, provided respectively at one end and the other end in the cylindrical reflector, and being penetrated by the ultraviolet lamp at the center thereof; and a copper sheet filter being an air permeable photocatalytic sheet filter having a diameter nearly equal to an inner diameter of a cylinder member and closing a lower end opening of the cylindrical reflector.

### TECHNICAL FIELD

The present invention relates to an air disinfection and cleaning device to disinfect and clean an air in, for example, a hospital room and a consultation room, and an exhaled gas disinfection and cleaning device, an interior air disinfection and cleaning device, and a simplified isolation device using the same, and relates specifically to a simple and compact air disinfection and cleaning device that can obtain high disinfection and cleaning effects with a small volume and a short total length, and an exhaled gas disinfection and cleaning device, an interior air disinfection and cleaning device, and a simplified isolation device using the same.

### BACKGROUND ART

One of the infection routes of infectious diseases is airborne infection. Airborne infection is to infect a third person by breathing in air floating bacteria dispersed from a patient into the respiratory organs. A general measure to prevent airborne infection is to isolate infected patients in private rooms or in cohort and to make a medical worker wear a mask when entering a room same as the infected patients. However, a mask is capable of preventing bacteria from invading into the respiratory organs at the time of wearing, while as long as there are bacteria in an interior air, it is impossible to perfectly prevent airborne infection.

For example, there is a case of using a mechanical ventilator to a critically ill patient of H5N1 (avian influenza) to be referred to have a mortality rate of 60%. However, conventional mechanical ventilators have exhausted an exhaled gas breathed out by a patient into a room without disinfection, so that there used to be a risk of airborne infection of the bacteria contained in an exhaled gas of a patient to medical workers and other patients.

To solve such a problem, Japanese Patent Application Kokai Publication No. 2001-198201 (Patent Document 1) proposes “a method of disinfection and inactivation by putting an ultraviolet disinfection lamp in an exhalation circuit of a mechanical ventilator to rotate exhalation around the disinfection lamp” (paragraph [0006] (ii)).

Although not a device disinfecting an exhaled gas, Japanese Utility Model Application Kokai Publication No. S 61-151738 (Patent Document 2) proposes a deodorizing device having a configuration of storing an electric lamp 1 generating ultraviolet rays in a cylindrical vent pipe 3 and attaching a spiral fin 2 with a metal oxide layer 4 applied thereon on an outer periphery of the electric lamp 1 (refer to FIG. 1). This deodorizing device is used for elimination of an odor derived from a non-oxidized sulfur compound of a fermentation gas, a human waste treatment plant, a livestock farm, and the like.

The metal oxide layer 4 in this deodorizing device is made with either titanium oxide or zinc oxide or a mixture thereof and is excited by receiving ultraviolet radiation from the electric lamp 1. When feeding a gas containing a non-oxidized sulfur compound along such metal oxide layer 4, the non-oxidized sulfur compound is oxidatively degraded to sulfur dioxide, sulfuric anhydride, carbon monoxide, water, and the like to eliminate an odor in the gas.
[Patent Document 1] Japanese Patent Application Kokai Publication No. 2001-198201
[Patent Document 2] Japanese Utility Model Application Kokai Publication No. S 61-151738

However, in Patent Document 1 described above, there is a description to disinfect an exhaled gas of a patient with an ultraviolet disinfection lamp, while there is no description at all of a specific configuration to carry out it, and thus it used not to be possible to achieve the above disinfection of an exhaled gas based on the description in Patent Document 1.

Since the deodorizing device in Patent Document 2 described above has a configuration of attaching the spiral fin 2 on an outer periphery of the electric lamp 1 generating ultraviolet rays, there used to be a problem that the fin 2 blocks ultraviolet rays to decrease an amount of ultraviolet radiation to the gas and thus effective disinfection cannot be carried out.

Therefore, to give a sufficient disinfection performance to the deodorizing device of Patent Document 2, it is considered to increase the volume of the vent pipe 3 and also to elongate the total length. However, in this case, the deodorizing device gets larger as a whole, which is no longer a size to fit into an exhalation circuit of a mechanical ventilator and becomes an excessively large scale device for disinfection of an exhaled gas for one patient.

### DISCLOSURE OF THE INVENTION

The present invention has been made in view of the above problems, and it is an object thereof to provide a simple and compact air disinfection and cleaning device that can obtain high disinfection and cleaning effects with a small volume and a short total length, and an exhaled gas disinfection and cleaning device, an interior air disinfection and cleaning device, and a simplified isolation device using the same.

### BRIEF DESCRIPTION OF THE DRAWINGS

FIG. 1 is a schematic view illustrating an air disinfection and cleaning device according to First Embodiment of the present invention and an exhaled gas disinfection and cleaning device using the same.

FIG. 2 is a partial cross-sectional perspective view illustrating an internal structure of the air disinfection and cleaning device of FIG. 1.

FIG. 3 is a partial cross-sectional view to illustrate disinfection and cleaning actions of the air disinfection and cleaning device of FIG. 1.

FIG. 4 is a schematic view illustrating an air disinfection and cleaning device according to Second Embodiment of the present invention and an exhaled gas disinfection and cleaning device using the same.

FIG. 5 is a schematic view illustrating an air disinfection and cleaning device according to Third Embodiment of the present invention.

FIG. 6 is a perspective view illustrating an interior air disinfection and cleaning device (Fourth Embodiment) using the air disinfection and cleaning device of FIG. 5

FIG. 7 is a perspective view illustrating a modification of the interior air disinfection and cleaning device.

FIG. 8 is a perspective view illustrating a simplified isolation device (Fifth Embodiment) using the air disinfection and cleaning device of FIG. 5.

FIG. 9 is a perspective view illustrating a simplified isolation device (Sixth Embodiment) using the air disinfection and cleaning device of FIG. 5.

FIG. 10 is a partial cross-sectional view illustrating an air disinfection and cleaning device according to Seventh Embodiment of the present invention.

### DETAILED DESCRIPTION OF THE INVENTION

### Overall Description

An air disinfection and cleaning device according to an embodiment of the present invention is configured with: a cylindrical reflector having a cylindrical inner surface in which at least an air flows from one end to another end, the cylindrical inner surface subjected to mirror finishing; a rod shaped ultraviolet lamp disposed at a center within the cylindrical reflector parallel to a long side; and photocatalytic sheet filters being in an air permeable sheet shape having a diameter nearly equal to an inner diameter of the cylindrical reflector, provided respectively at one end and the other end in the cylindrical reflector, having the center penetrated by the ultraviolet lamp.

According to the above configuration, by disposing the ultraviolet lamp parallel to the long side of the cylindrical reflector, the reflection efficiency of ultraviolet rays within the cylinder becomes good. In addition, by providing the air permeable photocatalytic sheet filters at one end and the other end in the cylindrical reflector, these photocatalytic sheet filters do not block the ultraviolet rays within the cylinder.

These configurations in cooperation enable to effectively disinfect the air flowing from one end to the other end of the cylindrical reflector with the ultraviolet rays directly radiated from the ultraviolet lamp and the ultraviolet rays reflected by the cylindrical reflector. In addition, the reflection efficiency of ultraviolet rays within the cylinder becomes good, so that the amount of ultraviolet radiation radiated from each photocatalytic sheet filter also increases, which enables to oxidatively degrade organic compounds and inorganic compounds contained in the air.

This enables to obtain a high disinfection effect even when the cylindrical reflector has a smaller volume and a shorter total length, and thus it becomes possible to attempt downsizing, simplification, and cost reduction of the device. In addition, the intense ultraviolet rays can photocatalytically activate the photocatalytic filters effectively, which enables to increase effects of disinfection, odor elimination, deodorizing, and elimination of a toxic gas of the air passing through each photocatalytic sheet filter. As a result, a convenient air disinfection and cleaning device is achieved that is suitable for, for example, disinfection and cleaning of an exhalation gas for one patient by connecting to a breathing circuit.

Preferably, it is configured that the cylindrical reflector is made with a cylinder member with one end being closed and another end being opened, and the one end of the cylinder member is provided with an air inlet and the opening at the other end is closed with an air permeable copper sheet filter having a diameter nearly equal to an inner diameter of the cylinder member.

According to the above configuration, by closing at least one end of the cylindrical reflector, it is enabled to avoid leakage and a direct view of ultraviolet rays from inside the cylindrical reflector, and thus the safety can be improved. For example, when the cylindrical reflector is arranged with one end above and with the other end below, leakage and a direct view of ultraviolet rays from inside the cylindrical reflector can be avoided.

In addition, by closing the opening at the other end of the cylindrical reflector with the copper filter, the copper ions contained in the copper filter exhibit a disinfection effect and also it is possible to prevent bugs from entering the cylindrical reflector.

Preferably, it is configured that the cylindrical reflector is made with a cylinder member with one end and another end being closed, the one end is provided with an air suction port and the other end is provided with an air exhaust port, at least one of these suction port and exhaust port is connected to a suction fan to forcibly distribute an air from one end to the other end of the cylinder member.

According to the above configuration, the interior air can forcibly be sucked in the cylindrical reflector by the suction fan to be disinfected and cleaned, which enables to significantly increase the range of application of the present air disinfection and cleaning device. For example, when applying the present air disinfection and cleaning device to a breathing circuit or a mechanical ventilator, since an exhalation gas flows in the present air disinfection and cleaning device due to a respiratory effort of a patient, the exhalation gas can be disinfected and cleaned even without a suction fan (a suction fan may also be provided depending on the suction power of the suction fan or the operation mode of a mechanical ventilator). Still for example, in a case of disinfecting and cleaning the interior air by the present air disinfection and cleaning device, a suction fan becomes essential to suck the interior air into the present air disinfection and cleaning device.

Preferably, it is configured that the cylindrical reflector is made with a cylinder member with one end and another end being closed, and a plurality of the cylinder members are coupled in series via a pipeline capable of air distribution, the cylinder member coupled at one end of the series and the cylinder member coupled at another end of the series are provided respectively with a suction port and an exhaust port, and at least one of these suction and exhaust ports is connected to a suction fan to forcibly distribute the air in the plurality of cylinder members coupled from one end to the other end of the series.

As described above, although the present air disinfection and cleaning device has a compact and simple configuration while exhibiting high disinfection capabilities, when a larger amount of air has to be disinfected in a short time, the above configuration may be employed. According to the above configuration, by coupling cylindrical reflectors with a small volume and a short total length in series, downsizing of the entire device can be attempted while the length for disinfection of the device can be elongated, which enables to significantly improve the disinfection effect. In addition, even when one of the ultraviolet lamps arranged respectively in the plurality of cylindrical reflectors is burnt out, for example, it has a fail safe effect capable of disinfecting with the ultraviolet lamps in other cylindrical reflectors.

Preferably, it is configured that the ultraviolet lamp configuring the air disinfection and cleaning device has an electrode unit shut off from an internal space of the cylindrical reflector and distribution of an ambient air to the electrode unit is enabled.

For example, there is a case of supplying high concentration oxygen to a patient in a breathing circuit or a mechanical ventilator, and in such a case, an exhalation gas having a high oxygen concentration flows into the cylindrical reflector of the present air disinfection and cleaning device. In general, in an environment having a high oxygen concentration, there is a risk of ignition of peripheral components due to a spark. With that, according to the above configuration, even in a case where an air having a high oxygen concentration flows into the cylindrical reflector, the electrode unit of the ultraviolet lamp is shut off from the internal space of the cylindrical reflector and also the oxygen concentration around the electrode unit is decreased by distributing an ambient air, so that the risk of ignition of a component of the device due to a spark in the electrode unit can be avoided.

Preferably, it is configured that the ultraviolet lamp is provided with an approximately U shaped fluorescent tube by bending or bridging a fluorescent tube.

According to the above configuration, by employing an approximately U shaped fluorescent tube for the ultraviolet lamp, the device can be downsized while significantly improving the disinfection performance of an air with ultraviolet rays. In addition, compared with a straight fluorescent tube, the approximately U shaped fluorescent tube can put together the electrode unit at either one of one end or the other end of the cylindrical reflector. This enables to attempt simplification of the configuration of the device. For example, when employing the approximately U shaped fluorescent tube for the ultraviolet lamp, the above described configuration of decreasing the oxygen concentration around the electrode unit may be provided at either one of one end or the other end of the cylindrical reflector.

Preferably, it is configured that the cylindrical reflector is made with a cylinder member with one end and another end being closed, the one end of the cylinder member is provided with an air inlet, and the other end of the cylindrical member is provided with an air outlet, and

a rectification mechanism regulating an air flow flowing into the cylinder from the inlet is provided closer to the one end of the cylindrical reflector than the photocatalytic sheet filter.

In the cylindrical reflector, the air flowing into the cylinder in a turbulent state can be regulated with the photocatalytic filter having a honeycomb structure into a laminar flow. This enables to make the air flow uniform within the cylinder and effectively carry out disinfection, odor elimination, deodorizing, and elimination of a toxic gas with the ultraviolet lamp and the photocatalytic sheet filter. Here, as a rectification mechanism to regulate the air flow, various types of rectification mechanism can be applied that is capable of making the air flow uniform, such as a fin, a grid, and a perforated plate, for example.

Preferably, it is configured that the rectification mechanism is the air permeable photocatalytic sheet filter having the diameter nearly equal to the inner diameter of the cylindrical reflector, and an entire sheet of the photocatalytic sheet filter is provided nearly evenly with a large number of air permeable holes regulating the air flow.

According to the above configuration, the rectification mechanism made with the photocatalytic sheet filter regulates the air flow flowing into the cylinder to a laminar flow with the large number of air permeable holes and also is photocatalytically activated by receiving the ultraviolet rays reflected by the inner surface of the cylindrical reflector, which enables to effectively carry out disinfection, odor elimination, deodorizing, and elimination of a toxic gas of the air.

Preferably, it is configured that the inlet and the outlet of the cylindrical reflector is closed with an air permeable copper sheet filter.

According to the above configuration, by closing the inlet and the outlet of the cylindrical reflector with the air permeable copper sheet filter, the copper ions contained in the copper filter exhibit disinfection effect and also it is possible to prevent bugs and foreign substances from entering into the cylindrical reflector. Particularly, the copper ions exhibit a high degerming effect against enteropathogenic Escherichia coli O-157 and Legionella.

Preferably, it is configured that an air permeable copper sheet filter is provided and a drain outlet to exhaust a water droplet retained near the copper filter to outside the cylinder is provided, both closer to the other end than the outlet of the cylindrical reflector.

According to the above configuration, even when the moisture in the air flowing into the cylindrical reflector is dew condensed and a water droplet is retained at the bottom of the cylindrical reflector, the water droplet can be degermed by the copper filter and can be exhausted from the drain outlet to outside the cylinder. Particularly, the degerming effect of the copper filter is exhibited even when the power supply of the device is turned off, so that the water droplet retained in the cylindrical reflector can securely be degermed. In addition, the water droplet can be exhausted to a safe place via the drain outlet.

Preferably, it is configured that a mechanism is provided to inform of an accumulated operating time or a time for replacement of the ultraviolet lamp.

According to the above configuration, a user can securely be informed of the life or a time for replacement (for example, from 5000 to 8000 hours for those having a longer life), visually not observable, of the ultraviolet lamp in the cylindrical reflector. As the informing mechanism, a time counter or a liquid crystal display device, for example, can be employed that announces an accumulated operating time and a time for replacement by numbers, characters, symbols, or figures, and a mechanism announcing the time for replacement with an LED lamp or the like may also be employed.

A first exhaled gas disinfection and cleaning device according to an embodiment of the present invention is configured with: the air disinfection and cleaning device; and a tube to distribute at least an exhalation gas from a patient, wherein the tube is connected to an inlet or a suction port of the air disinfection and cleaning device, and after disinfecting and cleaning the exhalation gas by the air disinfection and cleaning device, it is exhausted into a room.

The exhaled gas disinfection and cleaning device having the above configuration is an application example of the present air disinfection and cleaning device to a breathing circuit, and according to the present exhaled gas disinfection and cleaning device, only by connecting an exhalation gas exhausting tube configuring a breathing circuit to the present air disinfection and cleaning device, the exhaled gas of the patient can securely be disinfected and cleaned, which enables to prevent air pollution inside the room in which the patient is isolated.

In particular, in-hospital and domestic airborne infection can be prevented, and it enables to effectively inhibit the spread of infection using the exhaled gas disinfection and cleaning device for a patient infected with, for example, a highly infectious influenza virus (e.g. H5N1, H1N1, etc.).

A second exhaled gas disinfection and cleaning device according to an embodiment of the present invention is configured with: the air disinfection and cleaning device; a breathing circuit having a plurality of tubes coupled to distribute an inhalation gas to a patient and an exhalation gas from the patient; and a ventilator controlling supply of the inhalation gas to the patient and exhaust of the exhaled gas of the patient, wherein an exhalation gas exhaust port of the ventilator is connected to an inlet or a suction port of the air disinfection and cleaning device, and after disinfecting and cleaning the exhalation gas by the air disinfection and cleaning device, it is exhausted into a room.

The exhaled gas disinfection and cleaning device having the above configuration is an application example of the present air disinfection and cleaning device to a mechanical ventilator, and with such exhaled gas disinfection and cleaning device as well, only by connecting an exhalation gas exhaust port of the ventilator to the present air disinfection and cleaning device, the exhaled gas of the patient can securely be disinfected and cleaned, which enables to prevent air pollution inside the room in which the patient is isolated.

An interior air disinfection and cleaning device according to an embodiment of the present invention is configured with: the air disinfection and cleaning device provided with the suction fan; and a first air suction and exhaust panel forming an internal space, by stretching an air permeable sheet material over an opening front of a thin box shape frame, closed with the sheet material in the box shape frame and provided with a connection unit capable of distribution of an air in communication with the internal space, wherein the connection unit of the first air suction and exhaust panel is connected to the suction port of the air disinfection and cleaning device, an interior air is sucked into the air disinfection and cleaning device via the first air suction and exhaust panel, and after disinfecting and cleaning the exhalation gas by the air disinfection and cleaning device, it is exhausted into a room.

The interior air disinfection and cleaning device having the above configuration is an application example of the present air disinfection and cleaning device to an air cleaner and is suitable for, for example, a consultation room in a hospital. According to the present interior air disinfection and cleaning device, the interior air is sucked by the entire surface of the sheet material of the first air suction and exhaust panel by Pascal's principle. When such first air suction and exhaust panel is provided by standing it on the back of a patient in a consultation room, the exhaled gas breathed out by the patient during medical consultation can efficiently be sucked to be disinfected and cleaned, which enables to effectively prevent airborne infection from the patient to medical workers. Further, in a case where the patient and the medical workers wear a mask, the possibility of infection can be reduced significantly.

Preferably, it is configured that the room further provided with a second air suction and exhaust panel arranged at a predetermined interval and facing the first air suction and exhaust panel therein, and a connection unit of the second air suction and exhaust panel is connected to a blowing mechanism to form an air flow in one direction from the second air suction and exhaust panel to the first air suction and exhaust panel.

According to the above configuration, an air flow from the second air suction and exhaust panel to the first air suction and exhaust panel in one direction is formed, and it is possible not to allow the air in the vicinity of the first air suction and exhaust panel to flow towards the second air suction and exhaust panel. For example, by arranging the patient on the first air suction and exhaust panel side and arranging a medical worker on the second air suction and exhaust panel, it is enabled to prevent airborne infection from the patient to the medical worker more securely.

Preferably, it is configured that the blowing mechanism is the air disinfection and cleaning device connected to the first air suction and exhaust panel or the air disinfection and cleaning device separate from the one connected to the first air suction and exhaust panel, and the exhaust port of either of the air disinfection and cleaning devices is connected to the connection unit of the second air suction and exhaust panel to flow a disinfected and cleaned air from the second air suction and exhaust panel to the first air suction and exhaust panel.

According to the above configuration, a purified air that is disinfected and cleaned always flows between the first and second air suction and exhaust panels, which enables to quickly form and maintain an extremely sanitary space. This enables to further securely prevent airborne infection from a patient to medical workers.

A first simplified isolation device according to an embodiment of the present invention is configured with: the air disinfection and cleaning device provided with the suction fan; a building frame forming a framework of a closed space; one or a plurality of sheet materials covering the building frame to form the closed space in the building frame; and a connection unit capable of distribution of an air in communication with the space closed with the sheet material, wherein a disinfected and cleaned air is supplied to the space closed with the sheet material by connecting the connection unit to the exhaust port or the suction port of the air disinfection and cleaning device, or an air in the space closed with the sheet material is sucked to be disinfected and cleaned.

According to the above configuration, by forming a simplified isolation room with the building frames and the sheet material and supplying a disinfected and cleaned air from the air disinfection and cleaning device into the simplified isolation room, inside the simplified isolation room can be kept as a clean space. Further in this configuration, by attaching a filter having a high dust collection capability, such as an HEPA filter, to the air exhaust port of the air disinfection and cleaning device, it is enabled to eliminate dust, dirt, and microparticles from the disinfected and cleaned air supplied into the simplified isolation room to make the simplified isolation room as a biological clean room.

On the contrary to the above configuration, when the air in the simplified isolation room is sucked by the air disinfection and cleaning device to be disinfected and cleaned, air pollution in the room with the present simplified isolation device installed therein can be prevented by exhalation of the patient in the simplified isolation room, which enables to attempt prevention of airborne infection and odor elimination in the room.

Preferably, it is configured with an air suction and exhaust panel forming an internal space, by stretching an air permeable sheet material over an opening front of a thin box shape frame, closed with the sheet material in the box shape frame and provided with the connection unit capable of distribution of an air in communication with the internal space, wherein a disinfected and cleaned air is supplied into the space via the air suction and exhaust panel by using the air suction and exhaust panel for a ceiling of the closed space and connecting the connection unit of the air suction and exhaust panel to the exhaust port of the air disinfection and cleaning device.

According to the above configuration, a disinfected and cleaned air is supplied into the simplified isolation room from the entire surface of the ceiling made with the air suction and exhaust panel, which enables to efficiently make inside the simplified isolation room in an aseptic and odorless state and also to effectively maintain the aseptic and odorless state.

A second simplified isolation device according to an embodiment of the present invention is configured with: the air disinfection and cleaning device provided with the suction fan; an air suction and exhaust panel forming an internal space, by stretching an air permeable sheet material over an opening front of a thin box shape frame, closed with the sheet material in the box shape frame and provided with a connection unit capable of distribution of an air in communication with the internal space; a building frame forming a framework of a closed space; one or a plurality of sheet materials covering the building frame to form the closed space in the building frame; an opening in communication with the space closed with the sheet material; and an air permeable filter member blocking the opening, wherein the air suction port of the air disinfection and cleaning device is connected to the connection unit of the air suction and exhaust panel, the air suction and exhaust panel is disposed in the space closed with the sheet material, and an air in the space is sucked to be disinfected and cleaned.

According to the above configuration, by forming a simplified isolation room with the building frame and the sheet material and sucking an air in the simplified isolation room with the air disinfection and cleaning device to be disinfected and cleaned, air pollution in the room with the present simplified isolation device installed therein can be prevented, which enables to attempt prevention of airborne infection and odor elimination in the room. In present simplified isolation device, as an air in the simplified isolation room is sucked via the air suction and exhaust panel, inside the simplified isolation room becomes under negative pressure, and a same amount of a new air is supplied into the simplified isolation room via the filter member, and thus the ventilation in the simplified isolation room is carried out well.

Preferably, it is configured that the opening and the filter member are provided above the space closed with the sheet material and also the air suction and exhaust panel is disposed below the space closed with the sheet material, and the air in the space is sucked from below the space to supply a new air from above the space.

According to the above configuration, in the simplified isolation room made with the building frame and the sheet material, an air flow occurs in one direction from the filter member above to the air suction and exhaust panel below, and thus suction of a polluted air via the air suction and exhaust panel and the supply of a new air via the filter become carried out more smoothly.

### Effects of the Invention

According to an air disinfection and cleaning device of the present invention, it is enabled to effectively disinfect the air flowing from one end to the other end of the cylindrical reflector with the ultraviolet rays directly radiated from the ultraviolet lamp and the ultraviolet rays reflected by the cylindrical reflector. This enables to obtain a high disinfection effect even when the cylindrical reflector has a smaller volume and a shorter total length, and thus it becomes possible to attempt downsizing, simplification, and cost reduction of the device. In addition, the reflection efficiency of ultraviolet rays within the cylinder becomes good, so that the amount of ultraviolet radiation radiated from each photocatalytic sheet filter also increases, which enables to oxidatively degrade organic compounds and inorganic compounds contained in the air.

According to the exhaled gas disinfection and cleaning device of the present invention, only by connecting an exhalation gas exhausting tube of a breathing circuit or an exhalation gas exhaust port of the ventilator configuring a mechanical ventilator to the present air disinfection and cleaning device, the exhaled gas of the patient can securely be disinfected and cleaned, which enables to prevent air pollution inside the room in which the patient is isolated.

According to the interior air disinfection and cleaning device of the present invention, the interior air can be sucked by the entire surface of the sheet material of the first air suction and exhaust panel. When such first air suction and exhaust panel is provided by standing it on the back of a patient in a consultation room, the exhaled gas breathed out by the patient during medical consultation can efficiently be sucked to be disinfected and cleaned, which enables to effectively attempt to prevent airborne infection from the patient to medical workers and eliminate an interior odor.

According to the simplified isolation device of the present invention, by forming a simplified isolation room with the building frames and the sheet material and supplying a disinfected and cleaned air from the air disinfection and cleaning device into the simplified isolation room, inside the simplified isolation room can be kept as a clean space. On the contrary, when the air in the simplified isolation room is sucked by the air disinfection and cleaning device to be disinfected and cleaned, air pollution in the room with the present simplified isolation device installed therein can be prevented by exhalation of the patient in the simplified isolation room, which enables to attempt prevention of airborne infection and odor elimination in the room.

### DESCRIPTION OF ILLUSTRATED EMBODIMENTS

### First Embodiment

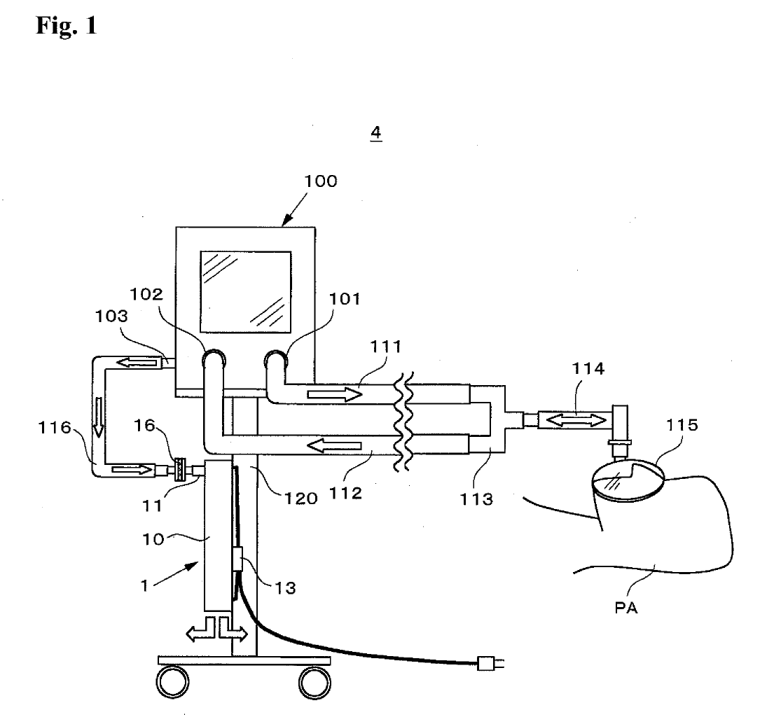

Firstly, a description is given to an air disinfection and cleaning device according to First Embodiment of the present invention and an exhaled gas disinfection and cleaning device using the same with reference to FIGS. 1 through 3.

In FIG. 1, an exhaled gas disinfection and cleaning device 4 according to the present embodiment comprises a ventilator 100 to control inhalation and exhalation of a patient PA, a stand 120 to place the ventilator 100 thereon, a breathing circuit to be a flow path of an inhalation gas and an exhalation gas, and an air disinfection and cleaning device 1 to disinfect and clean an exhaled gas of the patient PA.

### <Ventilator>

The ventilator 100 is connected to an oxygen blender, not shown. This oxygen blender mixes compressed air and compressed oxygen to generate an inhalation gas having an oxygen concentration from 21% (air) to 100% (pure oxygen). The ventilator 100 opens an inhalation valve, not shown, based on a decrease in the pressure due to a respiratory effort of the patient PA and feeds the inhalation gas generated by the oxygen blender from an inhalation gas tube connection port 101.

Meanwhile, an exhalation gas tube connection port 102 of the ventilation 100 is connected to an exhalation valve, not shown, to open the exhalation valve based on a rise in pressure due to a respiratory effort of the patient PA and take in an exhaled gas breathed out by the patient PA from the exhalation gas tube connection port 102 into the device. After that, the exhaled gas taken into the device is exhausted from an exhalation gas exhaust port 103 to outside the device.

### <Breathing Circuit>

To the inhalation gas tube connection port 101 and the exhalation gas tube connection port 102 of the ventilator 100, an inhalation gas tube 111 and an exhalation gas tube 112 are connected, respectively. Each of the tubes 111 and 112 are made with a corrugated tube of a synthetic resin. Each of the tubes 111 and 112 is connected to a Y connector 113 with one end split into two, and the other end of the Y connector 113 is connected to a catheter mount 114 made with a corrugated tube of a synthetic resin and a connection unit. The catheter mount 114 is connected to a mask 115, and the mask 115 is put on to cover the mouth and the nose of the patient PA. Each component of the breathing circuit described above is preferably disposable to be disposed for one time only.

### <Air Disinfection and Cleaning Device>

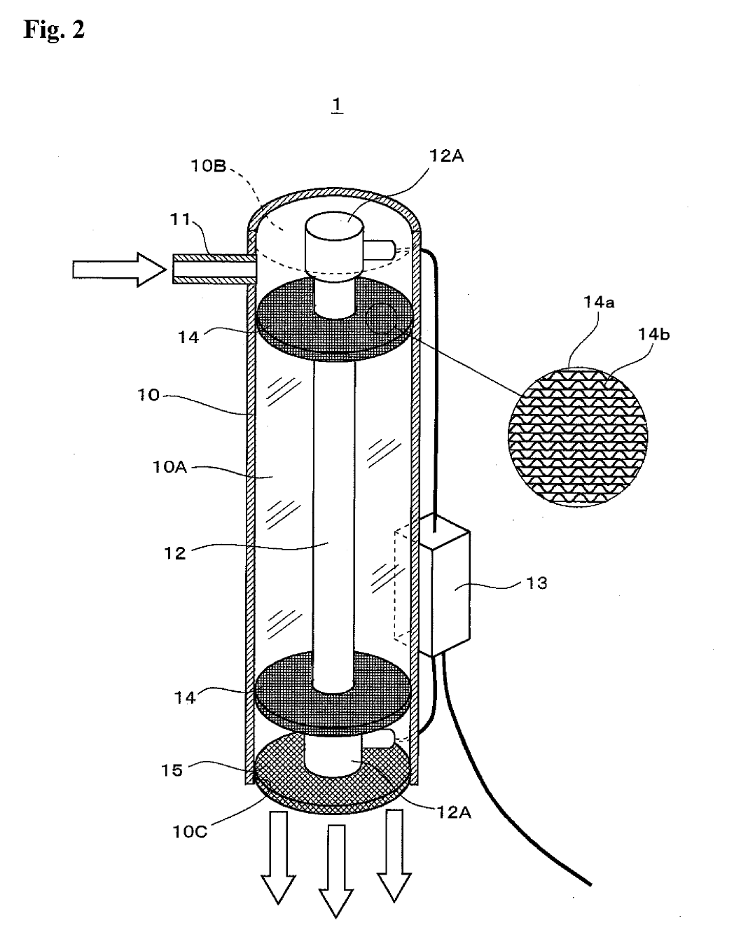

The exhalation gas exhaust port 103 of the ventilator 100 is connected to a disposable tube 116 made with a corrugated tube of a synthetic resin, and this tube 116 is connected to the air disinfection and cleaning device 1. As illustrated in FIG. 2, the air disinfection and cleaning device 1 has a configuration of storing an ultraviolet lamp 12, two photocatalytic sheet filters 14, 14, and one disk shaped copper filter 15 within a cylindrical reflector 10.

### <<Cylindrical Reflector>>

The cylindrical reflector 10 is a cylindrical member with an upper end as a closure 10B and a lower end as an opening 10C, and is configured with a cylindrical inner surface subjected to mirror finishing 10A. In the vicinity of the upper end of the cylindrical reflector 10, an inlet 11 for an exhalation gas is provided, and the inlet 11 is connected to the tube 116 via a dust filter 16 (refer to FIG. 1). An exhalation gas of the patient PA flowing in the tube 116 passes through the dust filter 16, flows in the cylindrical reflector 10 from the inlet 11, and flows out from the opening 10C at the lower end to outside the cylindrical reflector 10. Such cylindrical reflector 10 can be as small as, for example, to have an inner diameter from 100 to 150 mm approximately and a total length of 400 mm approximately.

### <<Ultraviolet Lamp>>

The ultraviolet lamp 12 is disposed at the center within the cylindrical reflector 10 parallel to the long side. Electrode units 12A, 12B at both ends of the ultraviolet lamp 12 are connected to a power supply unit (stabilizer) 13 provided outside the cylindrical reflector 10. As the power is supplied to the ultraviolet lamp 12 via the power supply unit 13, the ultraviolet lamp 12 is lit and ultraviolet rays are radiated within the cylinder. The ultraviolet rays are reflected by the mirror finishing 10A of the cylindrical inner surface.

### <<Photocatalytic Sheet Filters>>

The photocatalytic sheet filters 14, 14 are air permeable and in a disk shape having a diameter nearly equal to the inner diameter of the cylindrical reflector 10, are provided respectively at the upper and lower ends inside the cylindrical reflector 10, and the ultraviolet lamp 12 penetrates their center.

Each photocatalytic sheet filter 14 of the present embodiment is configured with a disk shaped substrate having a surface coated with a photocatalyst. As illustrated in the enlarged view in FIG. 2, the disk shaped substrate of the photocatalytic sheet filter 14 has a multilayer structure in which wavy interlinings 14b are bonded between straight liners 14a, and these liners 14a and interlinings 14b form a large number of horizontally lined up apertures (honeycombs). As the photocatalyst, titanium oxide can be used, and silver, activated carbon, or the like may also be blended into titanium oxide to enhance the disinfection or deodorant effect.

### <<Copper Sheet Filter>>

The copper sheet filter 15 is an air permeable mesh filter made by weaving a copper wire into a net or by sintering it into a non-woven fabric. By closing the opening 10C at the lower end of the cylindrical reflector 10 with the copper sheet filter 15, copper ions contained in the copper sheet filter 15 exhibit the disinfection effect and bugs can also be prevented from entering into the cylindrical reflector 10.

### <<Action of Air Disinfection and Cleaning Device>>

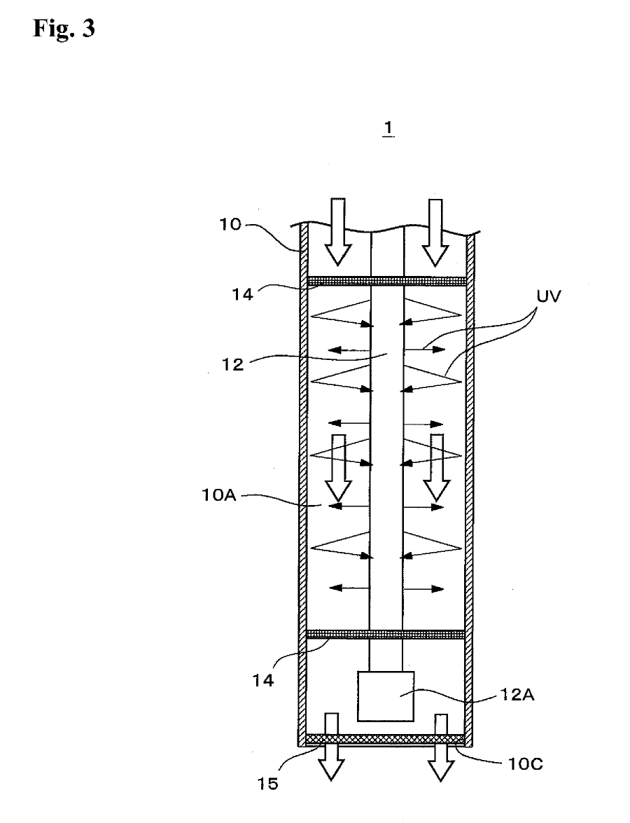

In the air disinfection and cleaning device 1 having the above configuration, as illustrated in FIG. 3, the ultraviolet rays radiated from the ultraviolet lamp 12 are reflected by the mirror finishing 10A applied to the entire inner surface of the cylindrical reflector 10 and thus reflection efficiency of the ultraviolet rays within the cylinder becomes greatest. As receiving the ultraviolet rays, both photocatalytic sheet filters 14, 14 located at the top and bottom of the cylindrical reflector 10 are activated.

In this state, as an exhaled gas of the patient PA flows in from the inlet 11 into the cylindrical reflector 10, the exhaled gas firstly passes through the photocatalytic sheet filter 14 at the upper end. At this time, the photocatalytic sheet filter 14 attaches an organic substance contained in the exhaled gas to a substrate surface to degrade the organic substance by the activated photocatalyst. This degrades a toxic substance causing unpleasant odors of the exhaled gas, and bacteria, mold, and the like in the air also perish.

The exhaled gas having passed through the photocatalytic sheet filter 14 at the upper end is exposed to the ultraviolet rays radiated directly from the ultraviolet lamp 12 and the ultraviolet rays reflected by the mirror finishing 10A of the cylindrical reflector 10 to be effectively disinfected without being blocked at all until reaching the photocatalytic sheet filter 14 at the lower end.

After that, the exhaled gas disinfected by the ultraviolet lamp 12 permeates through the photocatalytic sheet filter 14 at the lower end, and similar to above, is cleaned by the activated photocatalyst. Lastly, the exhaled gas is disinfected by the copper ions of the copper sheet filter 15 and is emitted from the opening 10C into the room.

### <<Effects of First Embodiment>>

According to the air disinfection and cleaning device 1 in the present embodiment having the above configuration, by disposing the ultraviolet lamp 12 parallel to the long side of the cylindrical reflector 10, the reflection efficiency of the ultraviolet rays within the cylinder becomes greatest. This enables to obtain high disinfection and cleaning effects even in a case where the cylindrical reflector 10 has a smaller volume and a shorter total length and thus to attempt downsizing, simplification, and cost reduction of the device.

Since the reflection efficiency of the ultraviolet rays within the cylinder becomes greatest, the amount of ultraviolet radiation radiated to each photocatalytic sheet filter 14 also increases. This enables to photocatalytically activate the photocatalyst effectively with intense ultraviolet rays and thus to increase disinfection and deodorizing of the exhaled gas passing through each photocatalytic sheet filter 14 and the effect of eliminating a toxic gas.

Further, according to the exhaled gas disinfection and cleaning device 4 in the present embodiment, only by connecting the air disinfection and cleaning device 1 to the exhalation gas exhaust port 103 of the ventilator 100, an exhaled gas of the patient can securely be disinfected and cleaned and it enables to prevent air pollution inside the room in which the patient is isolated.

In particular, in-hospital and domestic airborne infection can be prevented, and it enables to effectively inhibit the spread of infection using the exhaled gas disinfection and cleaning device 4 for a patient infected with, for example, a highly infectious influenza virus (e.g. H5N1, H1N1, etc.).

### EXAMPLE

A present air disinfection and cleaning device illustrated in FIG. 2 was manufactured to specifications in Table 1 below. For example, an amount of ultraviolet rays required for disinfection of 99.9% of influenza viruses is 6.6 mW·sec/cm². In contrast, the air disinfection and cleaning device of the present example is capable of radiating a disinfection dose of 66 mW·sec/cm² (ten times of the amount of ultraviolet rays required for disinfection of 99.9% of influenza viruses) in a cylindrical reflector having a diameter of 150 mm and an effective length of disinfection of 300 mm

TABLE 1

| | | |
|---|---|---|
| Cylindrical Reflector | Upper End | Closed |
| | Lower End | Opened |
| | Inner Diameter | 150 mm |
| | Total Length | 400 mm |
| Ultraviolet Lamp | Outer Diameter | 15 mm |
| | Total Length | 360 mm |
| | Effective Length of | 300 mm |
| | Disinfection | |
| | Ultraviolet | 10 mW/cm² |
| | Radiation Intensity | |
| | (Range of 10 cm | |
| | from Lamp Surface) | |
| Disc Shaped Titanium Oxide | Outer Diameter | 150 mm |
| Filter | Number of Sheets | 2 Sheets |
| Disc Shaped Copper Filter | Outer Diameter | 150 mm |
| | Number of Sheets | 1 Sheet |
| Amount of Ultraviolet Rays | | 66 mW · sec/cm² |
| in Cylinder | | |
| Effective Volume in Cylinder | | 52.45 Liters |
| Disinfectable Maximum Flow | | Approximately 50 |
| Rate | | Liters |

An acetaldehyde degradation test (deodorizing performance test) with a titanium oxide filter illustrated in FIG. 2 was performed in accordance with Table 2 below. The test results are shown in Table 3 below.

TABLE 2

| | | |
|---|---|---|
| Test | Sample | Titanium Oxide Filter |
| Conditions | Sample Dimensions | 60 × 40 mm × 5t |
| | Container | 5 Liters |
| | Target Gas | Acetaldehyde |
| | | (Carbon Dioxide) |
| | Ultraviolet Radiation Intensity | 15 mm |
| | (Example 1) | |
| | Ultraviolet Radiation Intensity | 360 mm |
| | (Example 2) | |
| | Ultraviolet Radiation Intensity | 300 mm |
| | (Example 3) | |
| | Ultraviolet Radiation Intensity | 10 mW/cm² |
| | (Example 4) | |

| | |
|---|---|
| Test Method | A sample and an ultraviolet lamp was put in a container |
| | and a target gas was injected. The ultraviolet lamp was |
| | lit to measure a residual concentration per unit time. |

TABLE 3

| Time (min) | 0 | 5 | 10 | 20 | 30 | 40 | 60 | 90 | 120 |
|---|---|---|---|---|---|---|---|---|---|
| Example 1 | 401 | 135 | 122 | 101 | 82 | 65 | 36 | 8.6 | 1.0 |
| (Acetaldehyde ppm) | | | | | | | | | |
| Example 2 | 401 | 135 | 126 | 111 | 96 | 81 | 54 | 24 | 7.2 |
| (Acetaldehyde ppm) | | | | | | | | | |
| Example 3 | 585 | 596 | 625 | 679 | 737 | 802 | 920 | 1077 | 1172 |
| (CO₂ ppm) | | | | | | | | | |
| Example 4 | 539 | 557 | 584 | 651 | 717 | 772 | 908 | 1080 | 1210 |
| (CO₂ ppm) | | | | | | | | | |

### <<Others>>

The air disinfection and cleaning device of the present invention is not limited to the embodiment described above. For example, the cylindrical reflector 10 is a cylindrical member in the above embodiment, while it may also be a polygonal tube member. Similarly, the external shapes of each photocatalytic sheet filter 14 and each copper sheet filter 15 can also be polygonal.

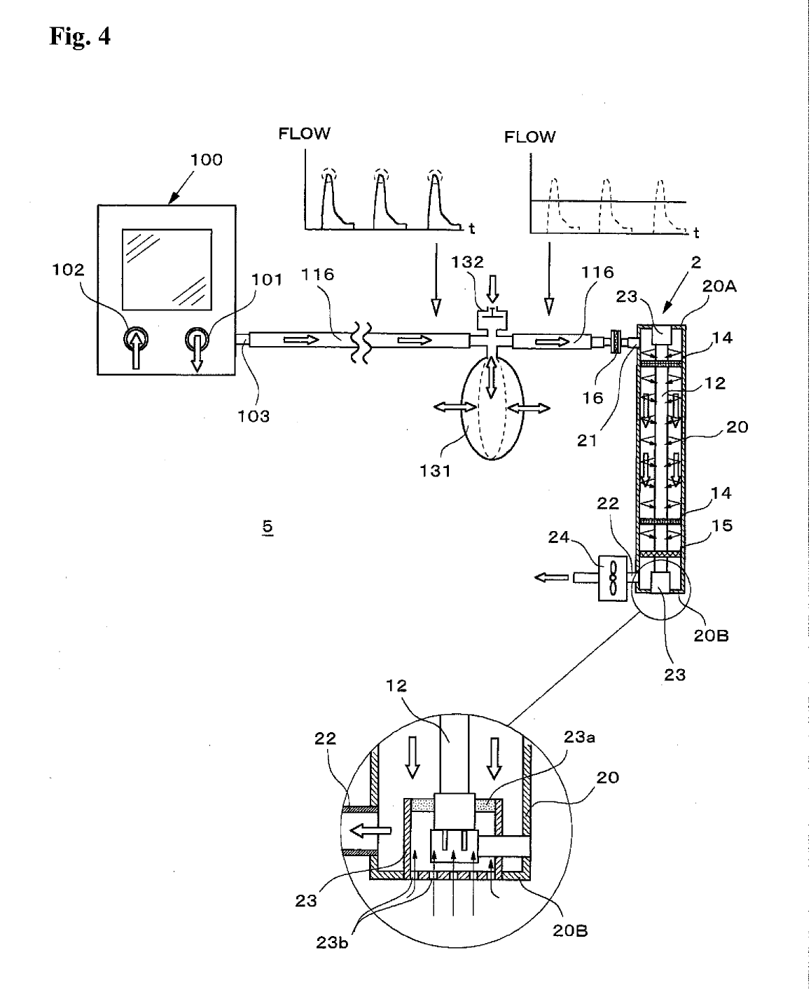

Although the lower end of the cylindrical reflector 10 is defined as the opening 10C, as illustrated in FIG. 4, both upper and lower ends of a cylindrical reflector 20 may also be closures 20A, 20B. Further, each of the upper and lower ends of the cylindrical reflector 10 is provided with one sheet of photocatalytic sheet filter 14 in the above embodiment, while photocatalytic sheet filter(s) may also be added between these filters 14, 14 as needed.

In addition, the embodiment described above exemplifies a configuration of applying the air disinfection and cleaning device 1 to a mechanical ventilator containing the ventilator 100, while the configuration is not limited to this and an exhaled gas of the patient PA can securely be disinfected and cleaned similar to above by connecting the air disinfection and cleaning device 1 to a breathing circuit through which the exhaled gas of the patient PA passes.

### Second Embodiment

Next, a description is given to an air disinfection and cleaning device according to Second Embodiment of the present invention and an exhaled gas disinfection and cleaning device using the same with reference to FIG. 4.

In FIG. 4, like components as in First Embodiment described above are referred to by an identical reference numeral and a detailed description is omitted. The inhalation gas tube connection port 101 and the exhalation gas tube connection port 102 of the ventilator 100 illustrated in this drawing are considered to be connected to a breathing circuit similar to that of First Embodiment described above and they are omitted from the illustration.

### <Flow Rate Adjustment of Exhalation Gas>

In FIG. 4, an exhaled gas disinfection and cleaning device 5 in the present embodiment is configured with an air disinfection and cleaning device 2 connected to the exhalation gas exhaust port 103 of the ventilator 100 via a reservoir bag 131 to maintain a constant flow rate of an exhalation gas passing through the air disinfection and cleaning device 2.

That is, as illustrated in a graph on the left of the reservoir bag 131 in FIG. 4, there are peaks (refer to broken line circles in the graph) in a flow of an exhaled gas breathed out by the patient PA, and there is a risk of insufficient disinfection and cleaning when an exhaled gas at the time of a peak expiratory flow passes through the air disinfection and cleaning device 2 extremely fast. With that, in the present embodiment, as illustrated in a graph on the right of the reservoir bag 131 in the drawing, a constant flow rate of an exhaled gas fed from the ventilator 100 is maintained to enable disinfection and cleaning of an exhaled gas more securely.

The reservoir bag 131 is a rubber made bag capable of expansion and contraction by flow in and flow out of an air, and the reservoir bag 131 is connected to a one way valve 132 that becomes in a closed state by receiving an air pressure from inside.

Meanwhile, the air disinfection and cleaning device 2 in the present embodiment has the cylindrical reflector 20 as a cylindrical member with the upper and lower ends to be the closures 20A, 20B, respectively. The cylindrical reflector 20 is provided with an air suction port 21 at the upper end and with an air exhaust port 22 at the lower end. The suction port 21 is connected to the reservoir bag 131 via the dust filter 16 and the tube 116. Meanwhile, the air exhaust port 22 is connected to a suction fan 24, and the suction fan 24 distributes an air at a constant rate from the upper end to the lower end of the cylindrical reflector 20.

In the exhaled gas disinfection and cleaning device 5 having the above configuration, as an exhalation gas is exhausted from the ventilator 100, the one way valve 132 becomes in a closed state by receiving the air pressure and the exhalation gas flows into the reservoir bag 131, and thus the reservoir bag 131 becomes in an expanded state. The exhalation gas accumulated in the reservoir bag 131 is sucked by the suction fan 24 of the air disinfection and cleaning device 2 to pass through the cylindrical reflector 20 at a constant rate.

By carrying out the above behaviors every time an exhalation gas is exhausted from the ventilator 100, a constant flow rate of an exhalation gas passing through the air disinfection and cleaning device 2 can always be maintained, which enables to disinfect and clean an exhalation gas more securely.

### <Measures for Oxygen Concentration in Exhalation Gas>

As described above, the ventilator 100 is connected to an oxygen blender, not shown, and can adjust an oxygen concentration in an inhalation gas within a range from 21% to 100%. Therefore, in a case of supplying an inhalation gas having a high oxygen concentration to the patient PA, the oxygen concentration in an exhalation gas also becomes high, and it is not generally considered that there is no possibility of ignition of peripheral components due to a spark.

With that, the air disinfection and cleaning device 2 in the present embodiment is configured with the respective electrode units 12A of the ultraviolet lamp 12 stored in electrode storage units 23, 23 to be shut off from an internal space of the cylindrical reflector 20 and also to enable distribution of an ambient air to each electrode unit 12A.

As illustrated in the enlarged view in FIG. 4, each electrode storage unit 23 is in a bottomed cylindrical shape with one opened end. Into a cylindrical opening of each electrode storage unit 23, a sealing member 23a formed with an elastic member, such as rubber, is press fitted. The sealing member 23a is tightly adhered to the outer periphery of the electrode unit 12A of the ultraviolet lamp 12 for sealing not to allow an exhalation gas in the cylindrical reflector 20 to enter the electrode storage unit 23. Meanwhile, at the cylindrical bottom of each electrode storage unit 23, a plurality of air permeable holes 23b, 23b, 23b . . . are drilled therein to enable distribution of an ambient air into the electrode storage unit 23.

According to such electrode storage unit 23, even when an exhalation gas having a high oxygen concentration flows into the cylindrical reflector 20, each electrode unit 12A of the ultraviolet lamp 12 can be shut off from the internal space of the cylindrical reflector 20. In addition, by distributing an ambient air in each electrode unit 12A of the ultraviolet lamp 12, the oxygen concentration around the electrode unit 12A can be decreased. This enables to avoid a risk of ignition of a component of the air disinfection and cleaning device 2 due to a spark of each electrode unit 12A.

### Third Embodiment

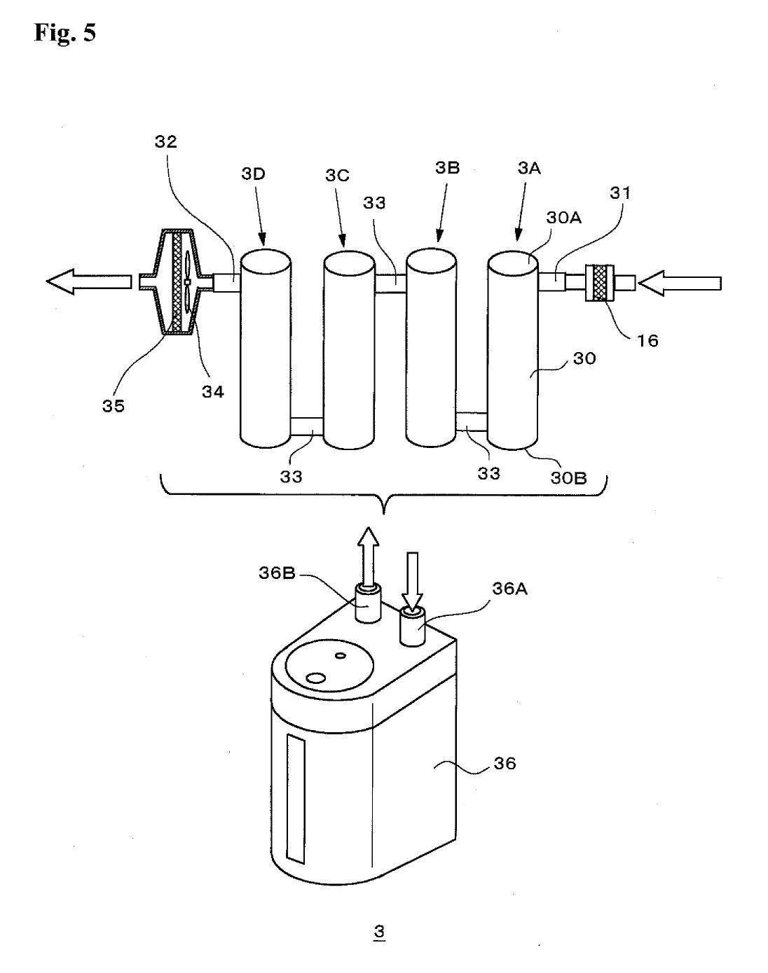

A description is given to an air disinfection and cleaning device according to Third Embodiment of the present invention with reference to FIG. 5.

As illustrated in FIG. 5, an air disinfection and cleaning device 3 in the present embodiment is configured with a plurality of air disinfection and cleaning devices 3A, 3B, 3C, 3D coupled in series, these air disinfection and cleaning devices 3A through 3D stored in a housing 36.

The cylindrical reflector 30 of each of the air disinfection and cleaning devices 3A through 3D is made with a cylindrical member with both upper and lower ends as closures 30A, 30B, and the upper and lower ends of the respective cylindrical reflectors 30 are coupled in series via pipelines 33, 33, 33 capable of air distribution. Although not shown, inside each cylindrical reflector 30, similar to the air disinfection and cleaning device 2 in FIG. 4, the ultraviolet lamp 12, the two photocatalytic sheet filters 14, 14, and one copper sheet filter 15 are stored.

Although the four air disinfection and cleaning devices 3A through 3D are illustrated in a horizontal line in FIG. 5 for the convenience of the description, the plurality of air disinfection and cleaning devices 3A, 3B, 3C, 3D . . . may also be aligned, in practice, collectively not to be bulky or aligned in a circular or arc form and then be coupled to each other with the pipelines 33.

The air disinfection and cleaning device 3A coupled at one end of the series is provided with a suction port 31, and the air disinfection and cleaning device 3D coupled at the other end of the series is provided with an exhaust port 32. The suction port 31 is connected to the dust filter 16, and by the dust filter 16, dust and dirt contained in an air sucked from outside are shut off from entering.

Meanwhile, the exhaust port 32 is connected to a suction fan 34 and an HEPA filter 35. The suction fan 34 forcibly distributes an ambient air sucked from the suction port 31 to each of the air disinfection and cleaning devices 3A through 3D. The HEPA filter (High Efficiency Particulate Air Filter) 35 is, as defined in Japanese Industrial Standards (JIS Z8122), “an air filter having particle collection efficiency of 99.97% or more relative to particles having a particle size of 0.3 μm at a rated flow and also having a performance of an initial pressure loss of 245 Pa or less”.

Even if it has failed to disinfect with each of the air disinfection and cleaning devices 3A through 3D by any chance, emission of the bacteria failed to be disinfected into the room can be inhibited securely by arranging the HEPA filter 35 on the exhaust side of the suction fan 34. In addition, each of the air disinfection devices 3A through 3D can eliminate dust, dirt, and microparticles, such as allergic substances like house dust and pollens from the disinfected and deodorized air.

Each of the air disinfection and cleaning devices 3A through 3D coupled in series are stored in the housing 36, and in the housing 36, the suction port 31 is coupled to an external suction port 36A of the housing 36 via the dust filter 16, and the exhaust port 32 is coupled to an external exhaust port 36B of the housing 36 via the suction fan 34 and the HEPA filter 35.

Although the air disinfection and cleaning devices 1, 2 in First and Second Embodiments described above exhibit high disinfection and cleaning capabilities while having a compact and simple configuration, the configuration of the present air disinfection and cleaning device 3 described above may be employed when, for example, a larger amount of air has to be disinfected and cleaned in a short time. According to the present air disinfection and cleaning device 3, coupling the air disinfection and cleaning devices 3A through 3D with a small volume and a short total length in series enables to significantly improve the disinfection and cleaning effects while attempting downsizing of the entire device.

In addition, even when one of the ultraviolet lamps 12, 12, 12 . . . arranged respectively in the plurality of cylindrical reflectors 30, 30, 30 . . . is burnt out, for example, it also has a fail safe effect capable of disinfecting with the ultraviolet lamp 12 in another cylindrical reflector 30.

The air disinfection and cleaning device 3 in the present embodiment is capable of disinfecting and cleaning an interior air by being installed in a room as is, and can be further applied to various modes as described in Fourth through Seventh Embodiments below.

### Fourth Embodiment

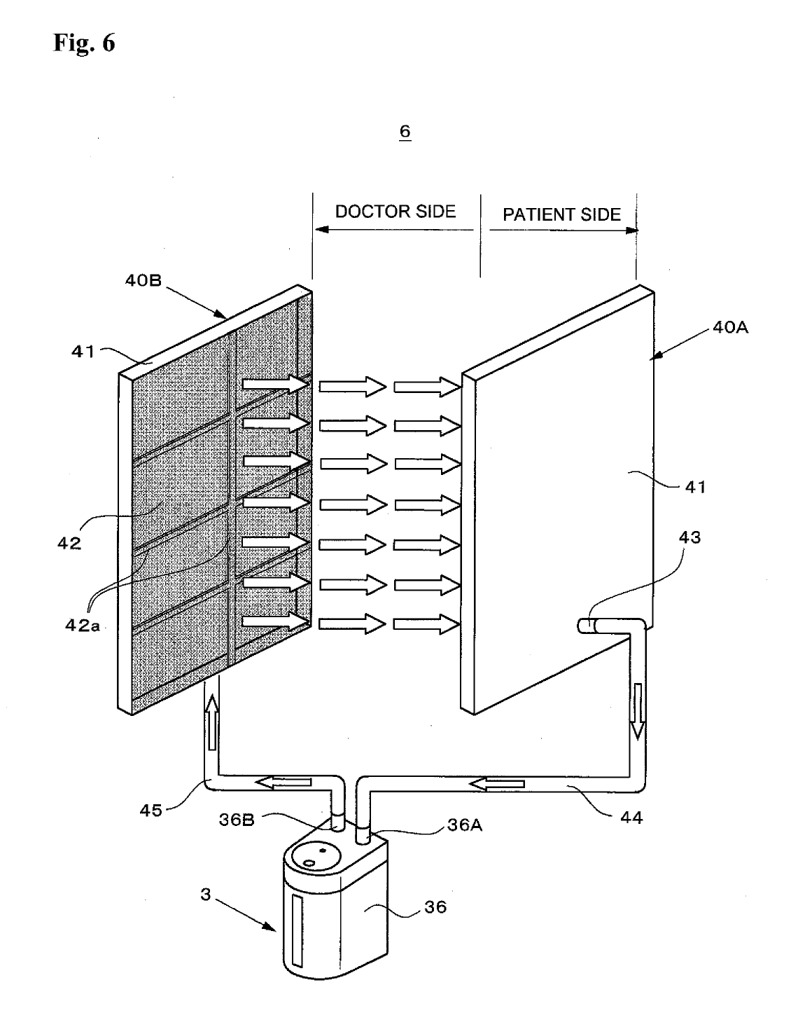

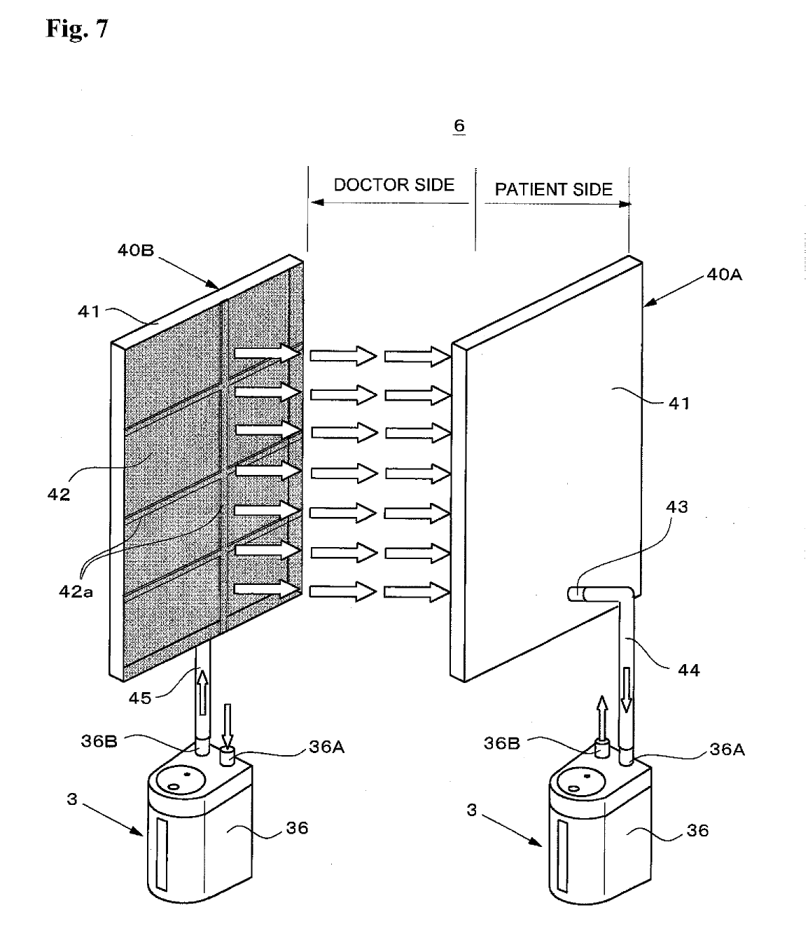

Next, a description is given to an interior air disinfection and cleaning device using the air disinfection and cleaning device in FIG. 5 as Fourth Embodiment of the present invention with reference to FIGS. 6 and 7. In FIGS. 6 and 7, like components as in Third Embodiment described above are referred to by an identical reference numeral and a detailed description is omitted.

In FIG. 6, an interior air disinfection and cleaning device 6 according to the present embodiment is configured, for example, to arrange facing first and second air suction and exhaust panels 40A, 40B like screens into a room, such as a consultation room in a hospital, to connect the external suction port 36A of the air disinfection and cleaning device 3 described above to the first air suction panel 40A, and to connect the external exhaust port 36B to the second air suction panel 40B.

Both the first and second air suction and exhaust panels 40A, 40B have an identical configuration of stretching an air permeable sheet material 42 on an opening front of a thin box shape frame 41. The air permeable sheet material 42 may be one at least capable of suction and exhaust of an air uniformly from the entire surface thereof by Pascal's principle, and for example, can use a non-woven fabric, a synthetic resin film with micro pores drilled in the entire surface thereof, or a nylon fiber sheet such as used for an air mattress of a comforter dryer.

Such air permeable sheet material 42 is fixed to a sheet frame 42a in a grid to prevent deformation at the time of sucking and exhausting an air. An internal space closed with the air permeable sheet material 42 is formed in the box shape frame 41, and a cylindrical connection unit 43 in communication with its internal space is projectingly provided on a back of the box shape frame 41.

The connection unit 43 of the first air suction and exhaust panel 40A is connected to the external suction port 36A of the air disinfection and cleaning device 3 via an air suction tube 44, and the connection unit 43 of the second air suction and exhaust panel 40B is connected to the external exhaust port 36B of the air disinfection and cleaning device 3 via an air exhaust tube 45.

The interior air disinfection and cleaning device 6 having the above configuration provides, for example in a consultation room in a hospital, the first air suction and exhaust panel 40A by standing it on the back of the patient and the second air suction and exhaust panel 40B on the back of a doctor. Then, a power supply of the air disinfection and cleaning device 3 is turned on and the ultraviolet lamp 12 is lit to activate the suction fan 34 (refer to FIG. 5).

The air on a patient side is then sucked uniformly from the entire surface of the air permeable sheet material 42 of the first air suction and exhaust panel 40A. The sucked air on the patient side is disinfected and cleaned by the air disinfection and cleaning device 3 (3A through 3D) through the air suction tube 44 and is exhausted uniformly from the entire surface of the air permeable sheet 42 of the second air suction and exhaust panel 40B through the air exhaust tube 45. This forms an air flow in one direction from the second air suction and exhaust panel 40B to the first air suction and exhaust panel 40A, which enables the air in the vicinity of the first air suction and exhaust panel 40A not to flow towards the second air suction and exhaust panel 40B, that is, not to allow the air on the patient side to flow towards the doctor.

According to the interior air disinfection and cleaning device 6 in the present embodiment, an exhaled gas breathed out by a patient during medical consultation can efficiently be sucked for disinfection and cleaning, which enables to effectively prevent airborne infection from the patient to the doctor and other medical workers. Further, when the patient and the medical workers wear a mask, the possibility of infection can be reduced significantly.

As illustrated in FIG. 7, the first and second air suction and exhaust panels 40A, 40B may also be connected to separate air disinfection and cleaning devices 3, 3, respectively. The air disinfection and cleaning device 3 connected to the first air suction and exhaust panel 40A sucks the air on the patient side from the entire surface of the first air suction and exhaust panel 40A to disinfect and clean it, and emits it from the external air exhaust port 36B into a consultation room. The disinfected and cleaned air is sucked from the external air suction port 36A of the air disinfection and cleaning device 3 connected to the second air suction and exhaust panel 40B, and after disinfected and cleaned again, is exhausted from the entire surface of the second air suction and exhaust panel 40B.

According to such configuration, an always disinfected and cleaned purified air flows between the first and second air suction and exhaust panels 40A, 40B, which enables to quickly form and maintain an extremely sanitary space. This enables to more securely prevent airborne infection from a patient to a doctor and the like.

### Fifth Embodiment

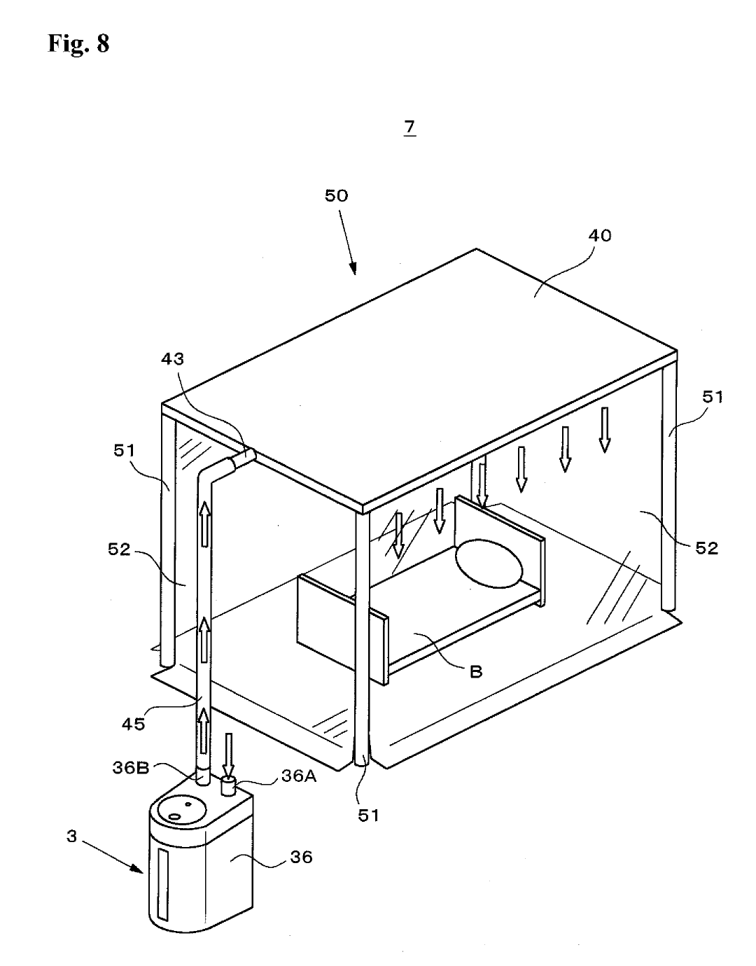

Next, a description is given to a simplified isolation device using the air disinfection and cleaning device in FIG. 5 as Fifth Embodiment of the present invention with reference to FIG. 8. In FIG. 8, like components as in Third and Fourth Embodiments described above are referred to by an identical reference numeral and a detailed description is omitted.

A simplified isolation device 7 according to the present embodiment can be used as a simplified biological clean room to prevent, for example, opportunistic infection of a leukemia patient, a burn patient, or the like. A biological clean room refers in general to a space controlled to have a predetermined degree or less of cleanliness for biological and non-biological microparticles in the room.

In FIG. 8, the simplified isolation device 7 in the present embodiment has a configuration in which a simplified isolation room 50 forming a closed space is connected to the air disinfection and cleaning device 3 to supply a disinfected and cleaned air into the isolation room 50. A framework of the isolation room 70 is made with a ceiling board and four columns, and for the ceiling board, an air suction and exhaust panel 40 having a configuration identical to that in FIG. 6 is used. The air suction and exhaust panel 40 supports the four corners with building frames 51, 51, 51, 51 taking the air permeable sheet material 42 facing downward. Non-air permeable sheets 52, 52, 52, 52 cover between these building frames 51, and thus a space closed from outside is formed. Such isolation room 50 is installed in a hospital room, and a bed B and medical equipment, not shown, are placed in the isolation room 50.

The air disinfection and cleaning device 3 has the external air exhaust port 36B connected to the connection unit 43 of the air suction and exhaust panel 40 via the air exhaust tube 45 and the external air suction port 36A opened to the room. As the air disinfection and cleaning device 3 is activated, the interior air is sucked from the external air suction port 36A into the air disinfection and cleaning device 3 to be disinfected and cleaned by the four air disinfection and cleaning devices 3A through 3D stored in the housing 36. Then, by passing through the HEPA filter 35 illustrated in FIG. 5, dust, dirt, and microparticles are eliminated from the disinfected and cleaned air. After that, a disinfected and cleaned clean air from which dust and dirt are eliminated is exhausted from the external air exhaust port 36B and exhausted from the entire surface of the air permeable sheet material 42 of the air suction and exhaust panel 40 through the air exhaust tube 45 into the isolation room 50.

According to the simplified isolation device 7 having such a configuration, the simplified isolation room 50 is formed with the air suction and exhaust panel 40, the building frames 51, and the air permeable sheet materials 52, and the biological and non-biological microparticles in the isolation room 50 can be controlled at a predetermined degree or less of cleanliness by supplying the disinfected and cleaned air from the air disinfection and cleaning device 3 into the isolation room 50, which enables to form an extremely simplified biological clean room. In addition, the air in the isolation room 50 can efficiently be circulated by supplying the disinfected and cleaned air from the entire surface of the ceiling board made with the air suction and exhaust panel 40 into the isolation room 50, and thus the biological clean room can be formed effectively and maintained.

### Sixth Embodiment

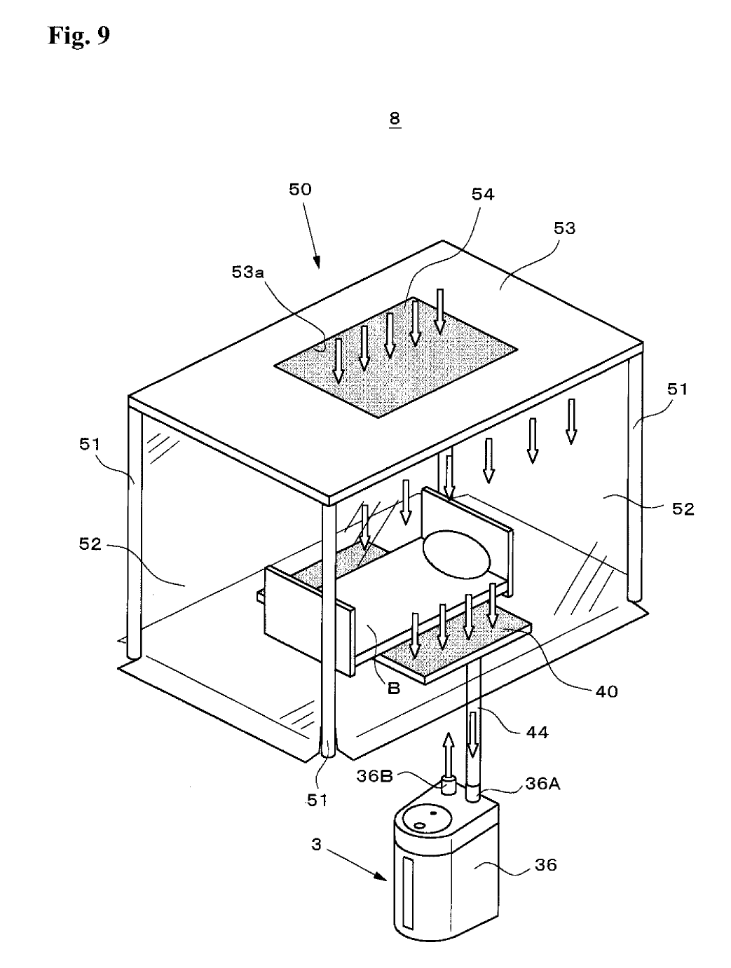

Next, a description is given to a simplified isolation device using the air disinfection and cleaning device in FIG. 5 as Sixth Embodiment of the present invention with reference to FIG. 9. In FIG. 9, like components as in Third through Fifth Embodiments described above are referred to by an identical reference numeral and a detailed description is omitted.

A simplified isolation device 8 according to the present embodiment can be used as, for example, a simplified isolation private room to prevent airborne infection to a third person by isolating a patient with an infection, such as tuberculosis, measles, chicken pox, H5N1, and H1N1.

In FIG. 9, the simplified isolation device 8 in the present embodiment forms the simplified isolation room 50 closed from outside by supporting four corners of a ceiling board 53 with the building frames 51, 51, 51, 51 and also covering between these building frames 51 with the non-air permeable sheets 52, 52, 52, 52. The ceiling board 53 of the isolation room 50 is provided with a rectangular opening 53a, and a filter member 54 is stretched over the opening 53.

These opening 53a and filter member 54 are intended to take an ambient air into the isolation room 50, and the filter member 54 has to be able to let an air smoothly flow into the isolation room 50 from outside and also to shut off dust and dirt from entering. As the filter member 54, it is possible to use those having relatively high air permeability, such as a prefilter for an air conditioner using a non-woven fabric as a material, for example.

On a floor (under the bed B in the drawing) in the isolation room 50, the air suction and exhaust panel 40 having a configuration identical to that in FIG. 6 is arranged with its air permeable sheet material 42 facing upward, and the air suction and exhaust panel 40 is connected to the external air suction port 36A of the air disinfection and cleaning device 3 via a suction tube 44. The air suction and exhaust panel 40 is desirably arranged, as illustrated in FIG. 9, in the vicinity of the infected patient and also in a position facing the filter member 54 of the ceiling board 53.

According to the simplified isolation device 8 having such a configuration, the simplified isolation room 50 is formed with the ceiling board 53, the building frames 51, and the non-air permeable sheet materials 52, and it is enabled to prevent airborne infection from an infected patient to a third person by sucking the air inside the isolation room 50 with the air disinfection and cleaning device 40 for disinfection.

In addition, when the air in the isolation room 50 is sucked via the air suction and exhaust panel 40, inside the isolation room 50 becomes under negative pressure and a same amount of a new air is supplied into the isolation room 50 via the filter member 54, and thus ventilation in the isolation room 50 is well carried out.

Further, since the air suction and exhaust panel 40 is arranged in the vicinity of the infected patient and also in a position facing the filter member 54 of the ceiling board 53 in the present embodiment, an air flow occurs in one direction from the filter member 54 above to the air suction and exhaust panel 40 below. This makes an exhaled gas of an infected patient smoothly sucked by the air suction and exhaust panel 40 and also the air polluted by bacteria does not flow out outside via the filter member 54.

### Seventh Embodiment

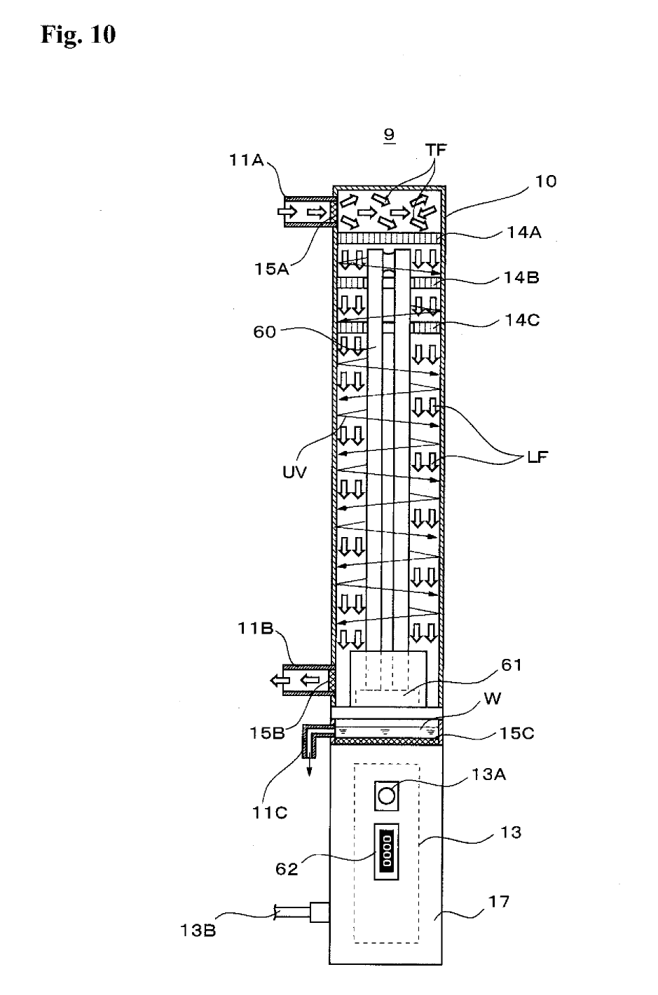

Next, a description is given to an air disinfection and cleaning device according to Seventh Embodiment of the present invention with reference to FIG. 10.

### <Cylindrical Reflector>

In FIG. 10, 9 denotes an air disinfection and cleaning device according to the present embodiment, having a configuration in which the cylindrical reflector 10 has closed one and the other ends. The one end of the cylindrical reflector 10 is provided with an air inlet 11A, and the other end is provided with an air outlet 11B. Similar to First Embodiment described above, the cylindrical reflector 10 is configured with the inner surface subjected to mirror finishing 10A, and reflection efficiency of ultraviolet rays exited from an ultraviolet lamp 60 described next becomes greatest.

### <Ultraviolet Lamp>

Within the cylinder of the cylindrical reflector 10, an ultraviolet lamp 60 provided with an approximately U shaped fluorescent tube is arranged. The ultraviolet lamp 60 in the present embodiment is configured with two fluorescent tubes bridged into an approximately U shape. Employment of such approximately U shaped ultraviolet lamp 60 enables to significantly improve the disinfection performance of an air by ultraviolet rays while downsizing the air disinfection and cleaning device 9.

Compared with the straight tube ultraviolet lamp 12 as illustrated in FIG. 2, the approximately U shaped ultraviolet lamp 60 can put together an electrode unit 61 at the other end of the cylindrical reflector 10, which enables to attempt simplification of the configuration of the air disinfection and cleaning device 9. For example, in a case of employing the approximately U shaped ultraviolet lamp 60, a configuration of decreasing the oxygen concentration around the electrode unit as illustrated in the enlarged view in FIG. 4 may be provided at the other end of the cylindrical reflector 10.

The ultraviolet lamp 60 with two fluorescent tubes bridged into an approximately U shape is employed in the present embodiment, while an ultraviolet lamp configured with one fluorescent tube bent into an approximately U shape may also be employed.

### <Rectification Mechanism: Photocatalytic Sheet Filter>

Near the inlet 11 within the cylinder of the cylindrical reflector 10, a disk shaped photocatalytic sheet filter 14A is disposed that is not penetrated by the ultraviolet lamp 60. Closer to the other end within the cylinder of the cylindrical reflector 10 than the photocatalytic sheet filter 14A, approximately annular photocatalytic sheet filters 14B and 14C that are penetrated by the ultraviolet lamp 60 are disposed at intervals. These photocatalytic sheet filters 14A through 14C have a configuration similar to the photocatalytic sheet filters 14 illustrated in FIG. 2 other than that the photocatalytic sheet filter 14A does not have a through hole.

That is, each of the photocatalytic sheet filters 14A through 14C are in an air permeable disk shape having a diameter nearly equal to the inner diameter of the cylindrical reflector 10 and having a configuration of coating a substrate surface with a photocatalyst. As illustrated in the enlarged view in FIG. 2, the substrate of each of the photocatalytic sheet filters 14A through 14C has a multilayer structure in which wavy interlinings 14b are bonded between straight liners 14a, and these liners 14a and interlinings 14b form a large number of horizontally lined up apertures (honeycombs). As the photocatalyst, titanium oxide can be used, and silver, activated carbon, or the like may also be blended into titanium oxide to enhance the disinfection or deodorant effect.

Here, as illustrated by white arrows in FIG. 10, the photocatalytic sheet filter 14A not having a through hole near the inlet 11 plays a role of a rectification mechanism regulating a turbulent flow TF of an air flowing into the cylinder from the inlet 11 to a laminar flow LF with the large number of apertures (honeycombs) aligned evenly in the substrate. After that, the air regulated with the photocatalytic sheet filter 14A passes through the photocatalytic sheet filters 14B and 14C having similar apertures (honeycombs) and uniformly flows within the cylinder while keeping the laminar flow LF. This enables to effectively carry out disinfection, odor elimination, deodorizing, and elimination of a toxic gas with the ultraviolet lamp 60 and the photocatalytic sheet filters 14A through 14C.

In addition, each of the photocatalytic sheet filters 14A through 14C attaches an organic substance contained in an air, such as an exhalation gas, to a substrate surface similar to First Embodiment described above to degrade the organic substance by an activated photocatalyst. This enables to degrade a toxic substance causing unpleasant odors of the exhaled gas, and bacteria, mold, and the like in the air perish.

Although the photocatalytic sheet filter 14A not having a through hole is employed as the rectification mechanism regulating the air flow in the present embodiment, it is not limiting. For example, various types of rectification mechanism capable of making the air flow uniform can be applied, such as a fin, a grid, and a perforated plate. It should be noted that, when the photocatalytic sheet filter 14A is employed as the rectification mechanism, in addition to the effect of regulating an air flow, effects of disinfection, odor elimination, deodorizing, and elimination of a toxic gas by the photocatalyst can also be obtained.

### <Copper Sheet Filter>

The air disinfection and cleaning device 9 in the present embodiment is configured with the inlet 11A and outlet 11B of the cylindrical reflector 10 and the bottom of the cylindrical reflector 10 at the other end provided with respective copper sheet filters 15A, 15B, 15C. Each of the copper sheet filters 15A through 15C is, similar to First Embodiment described above, an air permeable mesh filter made by weaving a copper wire into a net or by sintering it into a non-woven fabric.

By closing the inlet 11A and the outlet 11B of the cylindrical reflector 10 with the copper sheet filters 15A, 15B, copper ions contained in the copper sheet filters 15A, 15B exhibit the disinfection effect and bugs can also be prevented from entering into the cylindrical reflector 10. Particularly, copper ions exhibit a high degerming effect against enteropathogenic Escherichia coli O-157 and Legionella.

Meanwhile, when moisture contained in an air, such as an exhalation gas, is dew condensed within the cylinder and a water droplet W is retained at the bottom of the cylindrical reflector 10 at the other end, the copper sheet filter 15C provided at the bottom of the cylindrical reflector 10 at the other end degerms the water droplet W. In the vicinity of the bottom of the cylindrical reflector 10 at the other end, an exhaust port 11C is provided to exhaust the retained water droplet W outside the cylinder. Although not shown, it is possible to connect the exhaust port 11C to a hose to exhaust the water droplet W degermed by the copper sheet filter 15C to a safe place. Such a degerming effect of the copper sheet filter 15C is also exhibited when turning off the power supply of the air disinfection and cleaning device 9, so that the water droplet W retained in the cylindrical reflector 10 can be degermed securely.

### <Power Supply Unit, Time Counter>

The air disinfection and cleaning device 9 in the present embodiment is configured with a cylindrical housing 17 having an outer diameter same as that of the cylindrical reflector 10 is coupled to the cylindrical reflector 10 at the other end to store the power supply unit 13 inside the cylindrical housing 17. On the outer periphery of the cylindrical housing 17, a power switch 13A is disposed to carry out operation of turning on/off the power supply unit 13. Note that 13B denotes a power code to supply the power to the power supply unit 13.

Here, on the outer periphery of the cylindrical housing 17, a time counter 62 indicating an accumulated operating time of the ultraviolet lamp 60 is provided. By the time counter 62, a user can securely be informed of the life or the time for replacement, visually not observable, of the ultraviolet lamp 60 in the cylindrical reflector 10 (for example, from 5000 to 8000 hours for those having a longer life).

The mechanism to inform of the life or the time for replacement of the ultraviolet lamp 60 is not limited to the time counter 62, and for example, a liquid crystal display device announcing the accumulated operating time and the time for replacement of the ultraviolet lamp 60 by numbers, characters, symbols, or figures, a mechanism announcing the life and the time for replacement of the ultraviolet lamp 60 by an LED lamp, or the like may also be employed.

### Example

An air containing test bacteria (Staphylococcus aureus) is sucked by an air disinfection and cleaning device having a configuration are shown in FIG. 10, and a survival rate and a disinfection rate of the test bacteria after passing through this device were measured. As a result, according to the air disinfection and cleaning device of the present example, it was found that 36,000,000 of the test bacteria contained in 20 liters of the air can be disinfected 99.999997%.

TABLE 4

| Test Conditions | | Number of | | |
|---|---|---|---|---|
| | Measure- | Detected | Survival | Disinfection |
| | ment | Bacteria | Rate | Rate |
| Test Article | No. | (CFU/20 L-air) | (%) | (%) |
| Comparable | 1 | 23,000,000 | — | — |
| Example | 2 | 52,000,000 | — | — |
| (Ultraviolet | 3 | 61,000,000 | — | — |
| OFF) | 4 | 16,000,000 | — | — |
| | 5 | 27,000,000 | — | — |
| | Average | (A) 36,000,000 | — | — |
| | Value | | | |
| Example | 1 | 2 | — | — |
| (Ultraviolet | 2 | <1 | — | — |
| ON) | 3 | <1 | — | — |
| | 4 | <1 | — | — |
| | 5 | <1 | — | — |
| | Average | (B) 1.2 | 0.000003% | 99.999997% |
| | Value | | | |

Note that an ultraviolet lamp was used one having wattage of 36 W and an ultraviolet ray output of 12 W. The average value (B) of Example was calculated by defining the detection limit of the analyzer <1 as 1.

Survival Rate (%)=Average Value of Example (B)/Average Value of Comparable Example (A)×100%

Disinfection Rate (%)=100%−Survival Rate (%)

### REFERENCE NUMERALS

1, 2, 3 air disinfection and cleaning device
3A-3D air disinfection and cleaning device
4, 5 exhaled gas disinfection and cleaning device
6 interior air disinfection and cleaning device
7, 8 simplified isolation device
10 cylindrical reflector
10A mirror finishing
10B closure
10C opening
11 inlet
12 ultraviolet lamp
12A electrode unit
13 power supply unit
14 photocatalytic sheet filter
15 copper sheet filter
16 dust filter
20 cylindrical reflector
20A, 20B closure
21 air suction port
22 air exhaust port
23 electrode storage unit
23a sealing member
23b air permeable hole
24 suction fan
30 cylindrical reflector
30A, 30B closure
31 suction port
32 exhaust port
33 pipeline
34 suction fan
35 HEPA filter (High Efficiency Particulate Air Filter)
36 housing
36A external suction port
36B external exhaust port
40A first air suction panel
40B second air suction panel
41 box shape frame
42 air permeable sheet material
42a sheet frame
43 connection unit
44 air suction tube
45 air exhaust tube
40 exhaust panel
43 connection unit
50 isolation room
51 building frame
52 non-air permeable sheet
53 ceiling board
53a opening
54 filter member
100 ventilator
101 inhalation gas tube connection port
102 exhalation gas tube connection port
103 exhalation gas exhaust port
111 inhalation gas tube
112 exhalation gas tube
113 Y connector
114 catheter mount
115 mask
116 tube
120 stand
131 reservoir bag
132 one way valve
PA patient
UV ultraviolet
B bed
9 air disinfection and cleaning device
10 cylindrical reflector
11A inlet
11B outlet
11C exhaust port
13 power supply unit
13A power switch
13B power code
14A-14C photocatalytic sheet filter
15A-15C copper sheet filter
60 ultraviolet lamp
61 electrode unit
62 time counter
TF turbulent flow
LF laminar flow
W water droplet

1. An air disinfection and cleaning device, comprising:
a cylindrical reflector having a cylindrical inner surface in which at least an air flows from one end to another end, the cylindrical inner surface subjected to mirror finishing;
a rod shaped ultraviolet lamp disposed at a center within the cylindrical reflector parallel to a long side; and
photocatalytic sheet filters being in an air permeable sheet shape having a diameter nearly equal to an inner diameter of the cylindrical reflector, provided respectively at one end and the other end in the cylindrical reflector, having the center penetrated by the ultraviolet lamp.

2. The air disinfection and cleaning device of claim 1, wherein the cylindrical reflector is made with a cylinder member with one end being closed and another end being opened, and the one end of the cylinder member is provided with an air inlet and the opening at the other end is closed with an air permeable copper sheet filter having a diameter nearly equal to an inner diameter of the cylinder member.

3. The air disinfection and cleaning device of claim 1, wherein the cylindrical reflector is made with a cylinder member with one end and another end being closed, the one end is provided with an air suction port and the other end is provided with an air exhaust port, at least one of these suction port and exhaust port is connected to a suction fan to forcibly distribute an air from one end to the other end of the cylinder member.

4. The air disinfection and cleaning device of claim 1, wherein the cylindrical reflector is made with a cylinder member with one end and another end being closed, and a plurality of the cylinder members are coupled in series via a pipeline capable of air distribution, the cylinder member coupled at one end of the series and the cylinder member coupled at another end of the series are provided respectively with a suction port and an exhaust port, and at least one of these suction and exhaust ports is connected to a suction fan to forcibly distribute the air in the plurality of cylinder members coupled from one end to the other end of the series.

5. The air disinfection and cleaning device according to claim 1, wherein the ultraviolet lamp configuring the air disinfection and cleaning device has an electrode unit shut off from an internal space of the cylindrical reflector and distribution of an ambient air to the electrode unit is enabled.

6. The air disinfection and cleaning device according to claim 1, wherein the ultraviolet lamp is provided with an approximately U shaped fluorescent tube by bending or bridging a fluorescent tube.

7. The air disinfection and cleaning device according to claim 1, wherein the cylindrical reflector is made with a cylinder member with one end and another end being closed, the one end of the cylinder member is provided with an air inlet, and the other end of the cylindrical member is provided with an air outlet, and
a rectification mechanism regulating an air flow flowing into the cylinder from the inlet is provided closer to the one end of the cylindrical reflector than the photocatalytic sheet filter.

8. The air disinfection and cleaning device of claim 7, wherein the rectification mechanism is the air permeable photocatalytic sheet filter having the diameter nearly equal to the inner diameter of the cylindrical reflector, and an entire sheet of the photocatalytic sheet filter is provided nearly evenly with a large number of air permeable holes regulating the air flow.

9. The air disinfection and cleaning device of claim 7, wherein the inlet and the outlet of the cylindrical reflector is closed with an air permeable copper sheet filter.

10. The air disinfection and cleaning device according to claim 7, wherein an air permeable copper sheet filter is provided and a drain outlet to exhaust a water droplet retained near the copper filter to outside the cylinder is provided, both closer to the other end than the outlet of the cylindrical reflector.

11. The air disinfection and cleaning device according to claim 1, wherein a mechanism is provided to inform of an accumulated operating time or a time for replacement of the ultraviolet lamp.

12. An exhaled gas disinfection and cleaning device using the air disinfection and cleaning device according to claim 1, comprising:
the air disinfection and cleaning device; and
a tube to distribute at least an exhalation gas from a patient,
wherein the tube is connected to an inlet or a suction port of the air disinfection and cleaning device, and after disinfecting and cleaning the exhalation gas by the air disinfection and cleaning device, it is exhausted into a room.

13. An exhaled gas disinfection and cleaning device using the air disinfection and cleaning device according to claim 1, comprising:
the air disinfection and cleaning device;
a breathing circuit having a plurality of tubes coupled to distribute an inhalation gas to a patient and an exhalation gas from the patient; and
a ventilator controlling supply of the inhalation gas to the patient and exhaust of the exhaled gas of the patient,
wherein an exhalation gas exhaust port of the ventilator is connected to an inlet or a suction port of the air disinfection and cleaning device, and after disinfecting and cleaning the exhalation gas by the air disinfection and cleaning device, it is exhausted into a room.

14. An interior air disinfection and cleaning device using the air disinfection and cleaning device according to claim 3, comprising:
the air disinfection and cleaning device provided with the suction fan; and
a first air suction and exhaust panel forming an internal space, by stretching an air permeable sheet material over an opening front of a thin box shape frame, closed with the sheet material in the box shape frame and provided with a connection unit capable of distribution of an air in communication with the internal space,
wherein the connection unit of the first air suction and exhaust panel is connected to the suction port of the air disinfection and cleaning device, an interior air is sucked into the air disinfection and cleaning device via the first air suction and exhaust panel, and after disinfecting and cleaning the exhalation gas by the air disinfection and cleaning device, it is exhausted into a room.

15. The interior air disinfection and cleaning device of claim 14, wherein the room further provided with a second air suction and exhaust panel arranged at a predetermined interval and facing the first air suction and exhaust panel therein, and a connection unit of the second air suction and exhaust panel is connected to a blowing mechanism to form an air flow in one direction from the second air suction and exhaust panel to the first air suction and exhaust panel.

16. The interior air disinfection and cleaning device of claim 15, wherein the blowing mechanism is the air disinfection and cleaning device connected to the first air suction and exhaust panel or the air disinfection and cleaning device separate from the one connected to the first air suction and exhaust panel, and the exhaust port of either of the air disinfection and cleaning devices is connected to the connection unit of the second air suction and exhaust panel to flow a disinfected and cleaned air from the second air suction and exhaust panel to the first air suction and exhaust panel.

17. A simplified isolation device using the air disinfection and cleaning device according to claim 3, comprising:
the air disinfection and cleaning device provided with the suction fan;
a building frame forming a framework of a closed space;
one or a plurality of sheet materials covering the building frame to form the closed space in the building frame; and
a connection unit capable of distribution of an air in communication with the space closed with the sheet material,
wherein a disinfected and cleaned air is supplied to the space closed with the sheet material by connecting the connection unit to the exhaust port or the suction port of the air disinfection and cleaning device, or an air in the space closed with the sheet material is sucked to be disinfected and cleaned.

18. The simplified isolation device of claim 17, comprising an air suction and exhaust panel forming an internal space, by stretching an air permeable sheet material over an opening front of a thin box shape frame, closed with the sheet material in the box shape frame and provided with the connection unit capable of distribution of an air in communication with the internal space,
wherein a disinfected and cleaned air is supplied into the space via the air suction and exhaust panel by using the air suction and exhaust panel for a ceiling of the closed space and connecting the connection unit of the air suction and exhaust panel to the exhaust port of the air disinfection and cleaning device.

19. A simplified isolation device using the air disinfection and cleaning device according to claim 3, comprising:
the air disinfection and cleaning device provided with the suction fan;
an air suction and exhaust panel forming an internal space, by stretching an air permeable sheet material over an opening front of a thin box shape frame, closed with the sheet material in the box shape frame and provided with a connection unit capable of distribution of an air in communication with the internal space;
a building frame forming a framework of a closed space;
one or a plurality of sheet materials covering the building frame to form the closed space in the building frame;
an opening in communication with the space closed with the sheet material; and
an air permeable filter member blocking the opening,
wherein the air suction port of the air disinfection and cleaning device is connected to the connection unit of the air suction and exhaust panel, the air suction and exhaust panel is disposed in the space closed with the sheet material, and an air in the space is sucked to be disinfected and cleaned.

20. A simplified isolation device using the air disinfection and cleaning device of claim 19, wherein the opening and the filter member are provided above the space closed with the sheet material and also the air suction and exhaust panel is disposed below the space closed with the sheet material, and the air in the space is sucked from below the space to supply a new air from above the space.
